(19) United States
(12) Patent Application Publication
(10) Pub. No.: US 2007/0298091 A1
(43) Pub. Date: Dec. 27, 2007

(54) Transdermal Therapeutic System For Administering Analgesics

(75) Inventors: Heinrich KUGELMANN, Aachen (DE); Johannes Bartholomaeus, Aachen (DE)

Correspondence Address: CROWELL & MORING LLP;INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 11/851,645

(22) Filed: Sep. 7, 2007

Related U.S. Application Data
(63) Continuation of application No. PCT/EP2006/060484, filed on Mar. 6, 2006.

(30) Foreign Application Priority Data
Mar. 10, 2005 (DE) 10 2005 011 517.9

(51) Int. Cl. A61K 9/70

(52) U.S. Cl. 424/449

(57) ABSTRACT

Transdermal therapeutic systems for administering analgesics, preferably buprenorphine or one of its pharmaceutically acceptable salts or pro-drugs, and processes for the production of such systems.

### CROSS REFERENCE TO RELATED APPLICATIONS

This application is a continuation of international patent application no. PCT/EP2006/060484, filed Mar. 6, 2006 designating the United States of America and published in Germany on Sep. 14, 2006 as WO 2006/094961, which claims benefit to Federal Republic of Germany patent application no. DE 10 2005 011 517.9, filed Mar. 10, 2005, the entire disclosures of which are incorporated herein by reference.

### FIELD OF THE INVENTION

The invention relates to a transdermal therapeutic system for administering analgesics, preferably buprenorphine or one of its pharmaceutically acceptable salts or pro-drugs, and to a process for the production of such a system.

### BACKGROUND OF THE INVENTION

Active substances may be administered systemically through the skin using special transdermal therapeutic systems. Among the transdermal therapeutic systems (TTS) one can distinguish between membrane-controlled systems (reservoir TTS) and matrix-controlled systems (matrix TTS) as regards the liberation of the active substance.

A reservoir TTS is ordinarily a flat, self-adhesive pouch on the skin that contains the active substances in dissolved form. On the side facing the skin, the pouch is equipped with a semi-permeable membrane that controls the release of the active substances. The transport of the active substance into the bloodstream depends on the gradient of the chemical potential between the reservoir of active substance and the bloodstream (osmosis). A matrix TTS usually includes an adhesive film containing the active substance, which film will have an oval, round, or polygonal shape. The active substance is usually present in dissolved or suspended form in the adhesive film, from which it is released. F or further details reference is made, for example, to T. K. Gosh, Transdermal and Topical Drug Delivery Systems Es Into Practice, CRC Press, 1997; R. O. Potts et al., Mechanisms of Transdermal Drug Delivery (Drugs and the Pharmaceutical Sciences), Marcel Dekker, 1997; and R. Gurny et al, Dermal and Transdermal Drug Delivery. New Insights and Perspectives, Wissenschaftliche V G., Stuttgart, 1998.

In order for the active substance to act transdermally, it must diffuse through the epidermis of the skin at a sufficient rate and be absorbed in the bloodstream. An important limiting factor, in this case, is the variable permeability of the skin to different active substances. The transcutaneous permeation of the active substance from the transdermal therapeutic system may sometimes be improved, depending on the chemical structure and lipophilic nature of the active substance, by the addition of suitable adjuvants. The liberation of the active substance can be regulated by the composition and structure of the active substance matrix and/or diffusion membrane.

Besides having the desired pharmacokinetic parameters, a transdermal therapeutic system must be sufficiently well tolerated by the skin. On one hand, the ingredients should not cause allergies or contact dermatitis, while on the other hand, in particular, application and removal of the system should be gentle and not cause skin irritation. The degree of adhesion of the system to the skin, accordingly, must be sufficient to assure prolonged medication, but it should not be so great that it is not possible to remove it easily after application. Furthermore, a transdermal therapeutic system that is frequently used by a patient for a period of several days must display sufficient water resistance and adequate mechanical stability to prevent it from being damaged or inadvertently removed as a result of external factors.

One disadvantage of conventional transdermal therapeutic systems is that they frequently do not release the entire amount of the active substance present therein within the time interval recommended for application by the manufacturer. Thus, for example, about 2.5 μg/h of active substance are released continuously over a period of three days from a commercially available system having an area of 2.5 cm² and containing scopolamine as the active substance for the prevention of travel sickness and applied to the skin behind the ear. This adds up to only about 30% of the total amount of scopolamine present in the depot. Patches containing nitroglycerin that are available on the market and applied to the heart region of the chest are usually designed to release the active substance for 24 h. During this time interval, 5 and 10 mg, respectively, of active substance are continuously released. This is about 20% of the quantity of nitroglycerin present in the reservoir. For additional details, reference is made, for example, to K. H. Bauer et al., Lehrbuch der Pharmazeutischen Technologie, Wissenschaftliche V G, Stuttgart, 1999.

The quantity of active substance remaining in these transdermal therapeutic systems after proper use is not without its problems. On one hand, it causes additional costs, and on the other, it is frequently ecologically detrimental regarding the problem of efficient disposal of the systems.

Other problems arise when the active substance present in the transdermal therapeutic system has a potential for abuse, i.e. when it can be used contrary to specifications to achieve a euphoria-like state. For example, the opioid fentanyl is licensed for use in transdermal therapeutic systems under the drug laws of many countries (cf. K. A. Lehmann et al., Transdermal Fentanyl, Springer Verlag, 1991). However, other active substances with an abuse potential, such as morphine and buprenorphine, can also be administered transdermally.

Thus, for example, DE-A 39 39 376 discloses a transdermal therapeutic system containing buprenorphine as the active component. EP-A 413 034 discloses a laminate that has a layer of material impermeable or semi-permeable to moisture and two adhesive layers, of which at least one contains an active substance. The laminate can contain buprenorphine hydrochloride as the active substance. EP-A 484 543 discloses a patch consisting of a film layer having a thickness of from 0.5 to 4.9 μm and an adhesive layer containing the active substance. The patch can contain buprenorphine hydrochloride as the active substance. EP-A 1 174 137 discloses buprenorphine-containing patches that contain a certain adjuvant for transcutaneous administration of the active substance.

Transdermal therapeutic systems that contain buprenorphine or other strong analgesics are used, e.g., for pain therapy on cancer patients in the advanced stage of the disease so that the patient can remain at home and medicate himself. The transdermal therapeutic system assures a constant plasma concentration and a low risk of overdosage, contrary to other forms of administration. However, the disadvantage is that usually, after use, considerable quantities of the active substance remain in the system, often far above 50% of the quantity originally present, sometimes as much as from 85 to 90%, giving rise to additional problems.

Strong analgesics such as fentanyl, morphine and buprenorphine, are suitable not only for pain therapy but also for abusive practices, since a comparatively large residue of the analgesic remaining in the transdermal therapeutic system after use represents a potential source of intoxication for persons who intend to abuse it.

Because of their drug dependence, these persons display a sometimes astonishing creativity in the acquisition of new drugs. They do not normally require special equipment or special technical skills to extract the residue of the active agent from the transdermal therapeutic system. Thus, the removal of the active substance is often possible by simple dissolution thereof in pure ethanol, which is readily available, e.g., in drugstores.

Therefore, a need exists for transdermal therapeutic systems that are suitable for administration of analgesics and overcome the disadvantages of the transdermal therapeutic systems of the prior art.

### SUMMARY OF THE INVENTION

One object of certain embodiments of the invention is to provide a transdermal therapeutic system for the administration of analgesics that offers advantages over the transdermal therapeutic systems of the prior art. In particular, the transdermal therapeutic system should reduce the possibilities of abuse of the active substance present therein and assure appropriate adhesive properties and good pharmacokinetic parameters as regards the administration of the analgesic.

It has been found, surprisingly, that this problem can be solved by a transdermal therapeutic system for administering an analgesic, which includes

a carrier layer (T):

an adhesive layer (K) that immediately adjoins the carrier layer (T), is based on a pressure-sensitive adhesive, and contains the analgesic, and

optionally, a strippable protective layer (S),

the layer thickness of the adhesive layer (K) being less than 7.0 μm.

Surprisingly, the system of the invention displays excellent adhesive properties although the layer thickness of the adhesive film (K) is less than 7.0 μm and preferably in the range of from more than 2.0 μm to less than 5.0 μm. These excellent adhesive properties can even be achieved at a relatively high concentration of the analgesic in the adhesive layer (K), preferably at a concentration of the analgesic that is only slightly below its saturation concentration in the adhesive layer (K).

In the systems of the prior art, to date, it has been assumed that satisfactory adhesion of the system cannot be achieved with such small layer thicknesses of the adhesive layer (K), especially if at the same time the concentration of active substance in the adhesive layer is high.

Thus, for example EP-A 413 034 reveals that the adhesion of a patch to the skin is drastically reduced if the layer thickness of the adhesive film is less than 15 μm. According to this disclosure, at a layer thickness of 10μm or less it is difficult to achieve adhesion, if at all. EP-A 484 543 also discloses that when the thickness of the adhesive layer is reduced, the adhesion properties are impaired.

By contrast, the system of the present invention, in which the thickness of the adhesive layer (K) is less than 7.0 μm, preferably above 2.0 μm and below 5.0 μm, displays excellent adhesive properties while simultaneously permitting a high concentration of the analgesic in the adhesive layer (K).

As a measure of the adhesive properties of the system of the invention, preferably the force necessary to peel off a 4 cm wide system orthogonally from a flat stainless steel surface at a speed of 50 mm/min is measured. Usually in this case the measured values fluctuate with a certain range. The average adhesive force can be stated as the arithmetic mean of the measured maximal value and the measured minimal value. According to the invention, the measurement is performed as described in Example 9. The adhesive force thus determined on the system of the invention is preferably equal to 1.0 N/4 cm, more preferably at least 2.0 N/4 cm, even more preferably 3.5 N/4 cm, very preferably at least 5.0 N/4 cm and most preferably at least 10 N/4 cm.

In addition, with the aid of the system of the invention, it is possible, surprisingly, to release by far the major proportion of the analgesic originally present, within a specified period of use, usually within 1 to 5 days, so that after use only relatively small residues, if any, remain in the system. In this way, abuse of used systems is prevented or at least made very difficult, since any attempt to extract the (remaining) active substance from the system will be virtually unsuccessful.

The transdermal therapeutic system of the invention is characterized by good wearing comfort. Skin irritation when wearing is avoided, and the mechanical properties assure adequate resistance to external factors. Wearing comfort and resistance to mechanical external factors are matched to each other.

The thickness of the adhesive layer (K) of the system of the invention is less than 7.0 μm, preferably less than 6.5 μm, even more preferably less than 6.0 μm, very preferably less than 5.5 μm and most preferably less than 5.0 μm. Thus the layer thickness of the adhesive layer (K) is preferably greater than 2.0 μm. In a preferred variant, the thickness of the adhesive layer (K) is in the range of from more than 2.0 μm to less than 7.0 μm. More preferably, the thickness of the adhesive layer (K) is in the range of from 2.5 to 6.5 μm, even more preferably from 2.5 to 6.0 μm, very preferably from 2.5 to 5.5 μm and most preferably from 2.5 μm to less than 5.0 μm. It is highly preferred for the thickness of the adhesive layer (K) to be in the range of from more than 2.0 μm to less than 5.0 μm, particularly between 2.5 and 4.5 μm.

The thickness of the adhesive layer (K) can be determined by conventional methods. For example, the thickness of the adhesive layer (K) can be calculated from the total thickness of the system by subtracting the thickness of the carrier layer (T), of the protective layer (S) and of other layers optionally present besides the adhesive layer (K), and the total layer thickness of the system and the thickness of the individual layers can be measured, for example, using a micrometer screw or a laser.

The systems of the invention are characterized by a high homogeneity of the thickness of the adhesive layer (K). Since the analgesic is preferably uniformly distributed in the adhesive layer (K) in the system of the invention, the homogeneity of the thickness of the adhesive layer (K) is usually proportional to the homogeneity of the distribution of the analgesic. Accordingly, if adhesive present in one region of the adhesive layer (K) is less pressure sensitive than adhesive in another region, then less analgesic will be present at this site, and the homogeneity of the analgesic distribution can therefore serve as a measure of the homogeneity of the adhesive layer (K) thickness.

The interindividual variation of the thickness of the adhesive layer (K) when two systems of the invention are compared is preferably in the range of 100±5.0%, more preferably 100±4.0%, even more preferably 100±3.0%, very preferably 100±2.0% and most preferably 100±1.0%.

In order to determine this interindividual variation of the layer thickness of the adhesive layer (K), the analgesic contained in the two reference systems in the adhesive layer (K) can be quantified, for example, by extraction with the aid of a suitable solvent and execution of quantitative analysis according to methods reported in the pertinent pharmacopoeias for the analgesic in question, e.g. by UV spectroscopy. As a reference area, the entire adhesive layer (K) of the system of the invention may be used, but preferably only a part of the area of the adhesive layer (K) is studied and the variation of the layer thickness or variation in the quantity of analgesic is referred to this sub-area. The reference area preferably measures 10 cm², more preferably 5.0 cm² and even more preferably 1.0 cm². In the system of the invention it is particularly preferred for the variation of the quantity of analgesic relative to an area of 1.0 cm² is 100±5.0%, more preferably 100±4.0%, even more preferably 100±3.0%, very preferably 100±2.0% and most preferably 100±1.0%.

The transdermal therapeutic system of the invention contains an analgesic as the pharmaceutical active ingredient. Besides this analgesic, the system of the invention may also contain other active substances, e.g., other analgesics as well as active substances of other classes. The system of the invention preferably contains only one analgesic as the sole active ingredient.

According to the invention, the term analgesic preferably refers to those pharmaceutical substances that are assigned by the WHO to the ATC index N02 (preferably in the official German version of 1st January 2005, the substance of which is expressly incorporated herein by reference). Preferred analgesics are “opioids” (ATC code N02A), “other analgesics and antipyretics” (ATC code N02B) and “migraine remedies” (ATC code N02C). Particularly preferred within the group of preferred opioids are the “natural opium alkaloids” (ATC-Code N02AA), “phenylpiperidine derivatives” (ATC-Code N02AB), “diphenylpropylamine derivatives” (ATC-Code N02AC), “benzomorphane derivatives” (ATC-Code N02AD), “oripavine derivatives” (ATC-Code N02AE), “morphinan derivatives” (ATC-Code N02AF), “opioids in combination with spasmolytics” (ATC-Code N02AG), and “other opioids” (ATC-Code N02AX).

Thus, the transdermal therapeutic system of the invention preferably contains as the analgesic an opioid and more preferably an opioid selected from the group consisting of fentanyl, alfentanil, sufentanil, phenoperidine, anileridin, remifentanil, morphine, diacetylmorphine, hydromorphone, nicomorphine, oxycodone, diamorphine, ethylmorphine, ketobemidone, pethidine, dextromoramide, piritramide, dextropropoxyphene, bezitramide, methadone, pentazocine, phenazocine, buprenorphine, butorphanol, nalbufine, tilidine, dezocine, meptazinol, (1R,2R,4S)-2-[(dimethylamino)methyl-4-(p-fluorobenzyloxy)-1-(m-methoxyphenyl)-cyclohexanol, (1R,2R)-3-(2-dimethylaminomethylcyclohexyl)-phenol, (1R,2R)-3-(3-dimethylamino-1-ethyl-2-methylpropyl)-phenol, (1S,2S)-3(3-dimethylamino-1-ethyl-2-methylpropyl)-phenol, (2R,3R)-1 -dimethylamino-3 (3-methoxy-phenyl)-2-methyl-pentan-3-ol, (1RS,3RS,6RS)-6-dimethylaminomethyl-1-(3-methoxyphenyl)-cyclohexan-1,3-diol, preferably as the racemate, 3-(2-dimethylaminomethyl-1-hydroxy-cyclohexyl)-phenyl 2-(4-isobutylphenyl) propionate, 3-(2-dimethylaminomethyl-1-hydroxycyclohexyl)phenyl 2-(6-methoxy-naphthalen-2-yl) propionate, 3-(2-dimethylaminomethylcyclohex-1-enyl)-phenyl 2-(4-isobutyl-phenyl)propionate, 3-(2-dimethylaminomethyl-cyclohex-1-enyl)-phenyl 2-(6-methoxy-naphthalen-2-yl) propionate, (RR-SS)-2-acetoxy-4-trifluoromethylbenzoic acid 3-(2-dimethylaminomethyl-1-hydroxy-cyclohexyl) phenyl ester, (RR-SS)-2-hydroxy-4-trifluoromethylbenzoic acid 3-(2-dimethylaminomethyl-1-hydroxy-cyclohexyl) phenyl ester, (RR-SS)-4-chloro-2-hydroxybenzoic acid-3-(2-dimethylaminomethyl-1-hydroxycyclohexyl)phenyl ester, (RR-SS)-2-hydroxy-4-methylbenzoic acid 3-(2-dimethylaminomethyl-1-hydroxycyclohexyl) phenyl ester, (RR-SS)-2-hydroxy-4-methoxybenzoic acid, 3-(2-dimethylaminomethyl-1-hydroxycyclohexyl) phenyl ester, (RR-SS)-2-hydroxy-5-nitrobenzoic acid, 3-(2-dimethylaminomethyl-1-hydroxycyclohexyl) phenyl ester, (RR-SS)-2′,4′-difluoro-3-hydroxybiphenyl-4-carboxylic acid, 3-(2-dimethylaminomethyl-1-hydroxycyclohexyl) phenyl ester as well as the corresponding stereoisomeric compounds, in each case their corresponding derivatives, especially amides, esters, or ethers, and in each case their physiologically acceptable compounds, especially their salts and solvates, particularly preferably their hydrochlorides.

The opioid buprenorphine (ATC-Code N02AE01) is particularly preferred as the free base, in the form of one of its pharmaceutically acceptable salts, preferably buprenorphine hydrochloride, buprenorphine saccharinate, buprenorphine formate, buprenorphine mesylate, buprenorphine hydrogen citrate, buprenorphine nicotinate, or buprenorphine sebacinate, or in the form of one of its pharmaceutically acceptable prodrugs. Prodrugs of buprenorphine preferred according to the invention are its pharmaceutically acceptable esters or ethers.

The therapeutic system of the invention preferably contains the analgesic only in the adhesive layer (K); however, it is theoretically also possible that, in addition to the adhesive layer (K), other layers may be present that contain the analgesic or some other active substance. The adhesive layer (K) preferably contains the analgesic, preferably buprenorphine or one of its pharmaceutically acceptable salts or prodrugs, in a quantity of from 0.1 to 25 wt. %, more preferably from 1.0 to 20 wt. %, even more preferably from 2.5 to 17.5 wt. %, very preferably from 5.0 to 15 wt. % and most preferably from 9.0 to 12.5 wt. %, based on the total weight of the adhesive layer (K).

The concentration of the analgesic, preferably buprenorphine or one of its pharmaceutically acceptable salts or prodrugs, in the dried adhesive layer (K) is preferably only slightly below its saturation concentration. The saturation concentration is dependent, inter alia, on the chemical nature of the pressure-sensitive adhesive and can be determined by routine tests.

For example, a crystal of the analgesic may be applied to the surface of the previously produced, possibly dried adhesive layer (K) already containing the analgesic. If the adhesive layer (K) still absorbs the quantity of the analgesic applied as a crystal, then the concentration of the analgesic in the adhesive layer (K) is still below the saturation concentration. On the other hand, if the application of the crystal does not result in its absorption or even causes crystallization of the analgesic inside the adhesive layer (K), the crystal is acting, to a certain degree, as an inoculating crystal, indicating that the concentration of the analgesic is already at its saturation point. If the analgesic in the adhesive layer (K) is present in partially crystalline form, the saturation concentration has already been exceeded.

Alternatively, in a series of tests, adhesive layers (K) with progressively increased concentrations of the analgesic, e.g., in steps of 0.1 wt. % based on the dry weight of the adhesive layer (K) can be prepared. After the preparation of the adhesive layer (K), i.e. after drying and storing for a suitable time, e.g., 24 hours, if crystallization of at least part of the analgesic present therein can be observed, the saturation concentration of this sample has already been exceeded. The saturation concentration is thus reached in that sample having the analgesic concentration at which crystallization of the analgesic is just not observable.

According to the invention, the concentration of the analgesic in the dried adhesive layer (K) is preferably in the range of from 90% to 100% of the above-noted saturation concentration, more preferably from 92.4% to 100%, even more preferably from 95% to 100% and most preferably from 97.5% to 100%. It has been found, surprisingly, that excellent adhesion of the adhesive layer (K) can be achieved even at such a high concentration of the analgesic in the adhesive layer (K) even if the layer thickness of the adhesive layer (K) is below 7 μm, preferably in the range of from more than 2.0 μm to less than 5.0 μm.

The system of the invention preferably contains the active ingredient in a quantity that assures the liberation of one, two, three, four, or five daily doses within one, two, three, four, or five days. The system of the invention is particularly preferably designed for use over from 1 to 5 days, more preferably from 2 to 4 days and in particular 3 days, and contains the analgesic in an appropriate quantity and is preferably buprenorphine or one of its pharmaceutically acceptable salts or prodrugs. In the case of buprenorphine the rate of liberation is preferably from 35 to 70 μg/h, which can be regulated, inter alia, via the contact area of the transdermal therapeutic system with the skin.

The system of the invention preferably has a total area (skin contact area when properly used) of from 10 to 200 cm² and more preferably from 20 to 150 cm².

In a preferred embodiment, the system of the invention contains, in the adhesive layer (K), the analgesic, preferably buprenorphine or one of its pharmaceutically acceptable salts or prodrugs, in a quantity or more than 0.60, 0.65, 0.70, 0.75, 0.80, 0.85, 0.90, 0.95, 1.00, or 1.10 g/m². In another preferred embodiment, the system of the invention contains, in the adhesive layer (K), the analgesic, preferably buprenorphine or one of its pharmaceutically acceptable salts or prodrugs, in a quantity or more than 1.20, 1.30, 1.40, 1.50, 1.75, 2.0, 2.25, 2.5, or 3.0 g/m², based on the area of the adhesive layer (K). Experimental studies indicate, surprisingly, that, with the transdermal therapeutic system of the invention, the residue of the analgesic that remains behind after proper application of the transdermal therapeutic system, based on the quantity of analgesic originally present, i.e. before proper use, can be reduced if the concentration of the analgesic per unit area is more than 0.60 g/m² and preferably more than 0.80 g/m². However, this fact should not be construed as being tied to a scientific theory.

In the systems of the invention, the adhesive layer (K) immediately adjoins the carrier layer (T). The carrier layer (T) is preferably based on a polymer chosen from the group consisting of polyolefins, olefin-copolymers, polyesters, copolyesters, polyamides, copolyamides, polyurethanes, etc. Examples of suitable materials include, in particular, polyesters, preferably polyethylene terephthalates; polyolefins, preferably polyethylenes, polypropylenes, or polybutylenes; polycarbonates; polyethylene oxides; polyurethanes; polystyrenes; polyamides; polyimides; polyvinylacetates; polyvinylchlorides; polyvinylidene chlorides; copolymers, preferably acrylonitrile-butadiene-styrene terpolymers, or ethylene-vinyl acetate copolymers. The carrier layer (T) is especially preferably based on a polyester, especially on a polyethylene terephthalate, e.g. Hostaphan® RN.

In a preferred variant, the carrier layer (T) of the system of the invention has a layer thickness in the range of from 5.0 to 125 μm, more preferably from 10 to 115 μm, even more preferably from 25 to 100 μm, very preferably from 35 to 95 μm and most preferably from 50 to 85 μm.

In another preferred embodiment, the carrier layer (T) of the system of the invention has a layer thickness in the range of more than 125 μm, preferably in the range of from 125 to 2000 μm, more preferably from 130 to 1500 μm, even more preferably from 140 to 1000 μm and most preferably from 150 to 500 μm. It has been found that a transdermal therapeutic system cannot be felt directly by the patient, due to a loose sensation, when the total layer thickness is too small, which is regarded as being unpleasant by some patients. It may thus be advantageous to compensate for the reduction in total layer thickness, due to the production of extremely thin adhesive layers (K) as is the case with the system of the invention, by increasing the thickness of the carrier layer (T). The carrier layer (T) may in this case, for example, be made of a textile fabric or foamed material, which may contribute to the wearing comfort of the system of the invention. In addition, a relatively large layer thickness of the carrier layer (T) contributes to better mechanical stability of the system of the invention against external factors.

The ratio of the layer thicknesses of the carrier layer (T) to the adhesive layer (K) is preferably greater than 1.3:1, more preferably greater than 2.5:1, even more preferably greater than 5.0:1, very preferably greater than 7.5:1 and most preferably greater than 10:1.

In a preferred embodiment, the ratio of the layer thicknesses of the carrier layer (T) to the adhesive layer (K) is greater than 15:1, more preferably greater than 30:1, even more preferably greater than 50:1, very preferably greater than 75:1 and most preferably greater than 100:1.

As regards the layer thickness of the carrier layer (T) and the ratio of the layer thickness of the carrier layer (T) to the thickness of the adhesive layer (K) [(T):(K)], according to the invention the following combinations are also preferred.

| | Carrier layer (T) | (T):(K) |
|---|---|---|
| preferably | 5.0 to 125 μm | 2.5:1 to 40:1 |
| more preferably | 10 to 115 μm | 5.0:1 to 35:1 |
| even more preferably | 25 to 100 μm | 7.5:1 to 30:1 |
| very preferably | 35 to 95 μm | 10:1 to 25:1 |
| most preferably | 50 to 85 μm | 12.5:1 to 20:1 |

The carrier layer (T) preferably forms one of the two surface layers of the system of the invention, i.e. starting at the adhesive layer (K) directly adjoining the carrier layer (T), the transdermal therapeutic system of the invention preferably contains no additional layer beyond the carrier layer (T).

In an especially preferred embodiment, the transdermal therapeutic system of the invention consists of the carrier layer (T), the adhesive layer (K), and optionally the strippable protective layer (S). Particularly preferred variants of this version contain buprenorphine or one of its pharmaceutically acceptable salts or prodrugs as the analgesic and are summarized in the following table:

| | | | Most |
|---|---|---|---|
| | Preferably | More preferably | preferably |
| Layer thickness of the | 5.0-125 μm | 25-100 μm | 50-85 μm |
| carrier layer (T) | | | |
| Layer thickness of the | more than | more than 2.0 to | 2.5-4.5 μm |
| adhesive layer (K) | 2.0 to | less than 5.0 μm | |
| | less than | | |
| | 7.0 μm | | |
| Content of active | 0.1-25 wt. % | 2.5-17.5 wt. % | 9.0-12.5 wt. % |
| substance in the | | | |
| adhesive layer (K) | | | |

Other preferred variants are summarized in the following table:

| | | | Most |
|---|---|---|---|
| | Preferably | More preferably | preferably |
| Layer thickness of the | 125-2000 μm | 140-1000 μm | 150-500 μm |
| carrier layer (T) | | | |
| Layer thickness of the | more than | more than | 2.5-4.5 μm |
| adhesive layer (K) | 2.0 to | 2.0 to less | |
| | less than | than 5.0 μm | |
| | 7.0 μm | | |
| Content of active | 0.1-25 wt. % | 2.5-17.5 wt. % | 9.0-12.5 wt. % |
| substance in the | | | |
| adhesive layer (K) | | | |

In a particularly preferred embodiment, the system of the invention is such that after the application of the adhesive layer (K) to a suitable part of the skin of a patient, there will be released, within 72 hours, more than 20, 25, or 30 wt. %, preferably more than 35, 40, or 45 wt. %, even more preferably more than 50, 55, or 60 wt. %, very preferably more than 65, 70, or 75 wt. % and most preferably more than 80, 85, 90, or 95 wt. % of the quantity of analgesic originally contained in the adhesive layer (K), preferably buprenorphine or one of its pharmaceutically acceptable salts or prodrugs.

The quantity of active ingredient released can be determined by routine tests, optionally by using hairless rats. For example, after 72 hours the system can be removed from the skin and the residual quantity contained in the system extracted and quantified according to the procedures described for the analgesic in question in the relevant pharmacopoeias (e.g., US Pharmacopoeia, European Pharmacopoeia, Japanese Pharmacopoeia). If the analgesic is buprenorphine or one of its pharmaceutically acceptable salts or prodrugs, then for further details reference may be made, for example, to H. C. Evans, Drugs. 2003, 63(19):1999-2010; J. Sorge, Clin Ther. 2004, 26(11):1808-20; and R. Likar et al., Anesth Analg.2005, 100(3):781-5.

The liberation of buprenorphine can alternatively be determined in vitro by using a Franz diffusion cell, preferably FDC-400, using hairless mouse skin, as described for example in H. Imoto et al., Biol Pharm Bull. 1996, 19(2):263-7 and in DE-A 102 37 056, which are fully included herein by reference. In this case, the preferred embodiments of the system of the invention relate to the percentages given above for liberation after 72 hours. Details on the measurement of the transdermal permeation with the aid of a Franz diffusion cell are described, inter alia, in T. J. Franz, J. Invest. Dermatol. 64, 190-195 (1975).

The adhesive layer (K) of the system of the invention is based on a pressure-sensitive adhesive. Pressure sensitive adhesives (PSA) are well known to the person skilled in the art. In this connection, for example, reference may be made to I. Benedek, Pressure-Sensitive Adhesives and Applications, Marcel Dekker, 2nd edition, 2004. The pressure sensitive adhesive on which the adhesive layer (K) is based contains a polymer or a plurality of polymers selected from the group consisting of polyacrylates, polyvinyl ethers, polyvinyl alcohol, polyisobutylenes, acrylate copolymers, ethylene vinyl acetate copolymers, styrene-isoprene copolymers, styrene-butadiene copolymers, silicones and hydrogenated esters of colophony. Particular preference is given to acrylate-vinyl acetate copolymers that are available on the market, for example, under the trade name DURO-TAK®. These are very preferably used in combination with polyacrylates, e.g., Carbopol®, particularly Carbopol® 980 or with hydroxypropylcellulose as viscosity enhancers.

To improve the permeation of the analgesic present in the system of the invention, preferably buprenorphine or one of its pharmaceutically acceptable salts or prodrugs, the system of the invention preferably contains one or more adjuvants. In this connection, for example, reference may be made to D. S. Hsieh, Drug Permeation Enhancement: Theory and Applications (Drugs and the Pharmaceutical Sciences: a Series of Textbooks and Monographs), Marcel Dekker, 1994 and E. W. Smith et al., Percutaneous Penetration Enhancers, CRC Press, 1995.

The adhesive layer (K) preferably contains at least one auxiliary for improving the transdermal permeation of the analgesic, preferably of buprenorphine or one of its pharmaceutically acceptable salts or prodrugs, said adjuvant being selected from the group consisting of surfactants {e.g., nonionic surfactants, amphoteric surfactants, anionic surfactants, cationic surfactants, etc. including fatty acid esters, fatty alcohols, polyoxyethylene-cured castor oil (HCO), such as HCO-10, HCO-40, HCO-50, HCO-60; polysolvate (Tween®), such as Tween®-60, Tween(®-65, or Tween®-80; sorbitan esters, such as sorbitan trioleate, sorbitan monopalmitate, sorbitan monolaurate, sorbitan sesquioleate, polyoxyethylene/polyoxypropylene-sorbitan-mono-fatty acid esters, sorbitan-polyoxyethylene(160)-polyoxypropylene(30)-glycol-monostearate; glycerol esters such as glycerol monostearate, triacetin; benzalkonium chloride; benzetonium chloride}; amines {e.g. monoethanolamine, diethanolamine, triethanolamine, diisopropanolamine, triisopropanolamine}; inorganic bases {e.g. NaOH, KOH, Ca(OH)₂, NaHCO₃}; polyvinyl pyrrolidone; propylene glycol; benzyl alcohol; menthol; isosorbide nitrate; dodecyl azacycloheptan-2-one; lactic acid; and ethanol.

In another preferred embodiment, the system of the invention contains, in the adhesive layer (K), no adjuvant for improving the percutaneous permeation of the analgesic, preferably no auxiliary at all.

In a preferred embodiment, the transdermal therapeutic system of the invention contains a strippable protective layer (S). The latter is preferably silanized and/or fluoridated on at least the surface that faces the adhesive layer (K) and is removed from the system before use. As a result, the surface of the adhesive layer (K) is exposed. The protective layer (S) is preferably disposed directly adjacent to the adhesive layer (K).

Suitable materials on which the protective layer (S) can be based are known to the person skilled in the art. The protective layer (S) is preferably based on at least one material selected from the group consisting of polyester, polypropylene, polyvinyl chloride, aluminum, and paper, in which case at least the side adjacent to the adhesive layer (K) preferably displays a silicone and/or polyethylene and/or fluorosilicone and/or polytetrafluoroethylene coating. The carrier layer (T) is preferably based on a polyester, especially on Hostaphan® (RNT). The protective layer (S) preferably has a thickness of from 10 to 100 μm and more preferably from 25 to 50 μm.

The transdermal therapeutic system of the invention is preferably designed as a patch containing the active ingredient, in which the liberation of the active ingredient is matrix-controlled.

The system of the invention may contain the usual auxiliaries and additives, such as lipophilic additives, e.g., fat-soluble vitamins, unsaturated fatty acids, camphor, evening primrose oil, borage oil, St. John's Wort oil, fish oils, cod liver oil, ceramides, as well as dyes, plasticizers, solvents, skin-smoothing agents, and scents. In this connection, for example, one may refer to H. P. Fiedler, Lexikon der Hilfsstoffe für Pharmazie, Kosmetik und angrenzende Gebiete, Editio Cantor Aulendorff, 2002.

Another aspect of the invention relates to a process for the production of a transdermal therapeutic system as described above, including the steps:

(a) mixing the components of the adhesive layer (K), if required, in a suitable solvent;

(b) applying the mixture obtained in step (a) to one of the two surfaces of the carrier layer (T);

(c) if necessary, drying the mixture applied in step (b); and

(d) optionally applying the protective layer (S) to the mixture applied in step (b) and optionally dried in step (c).

It has been found, surprisingly, that the process of the invention can produce relatively thin adhesive layers (K) with a relatively high concentration of the analgesic therein while still achieving good adhesion properties. The layer thickness of the adhesive layer (K) of the systems produced by the process of the invention also has excellent homogeneity.

In a preferred embodiment of the process of the invention, the mixture is applied in step (b) with a film-drawing device, in which case the gap width of the blade is preferably in the range of from 10 to 60 μm, more preferably from 15 to 45 μm, even more preferably from 20 to 40 μm and very preferably from 25 to 35 μm and the gap width of the blade is very particularly 30 μm.

Suitable film-drawing devices are known to the person skilled the art. For example, an Erichsen Coatmaster 509/MC-1 is a suitable film-drawing device.

In the process of the invention, the application rate of the mixture in step (b) is preferably in the range of from 1.0 to 25 mm/s, more preferably from 2.0 to 10 mm/s, even more preferably from 3.0 to 7.0 mm/s and very preferably from 4.0 to 6.0 mm/s, while, the application rate is most preferably 5.0 mm/s.

The rate of application is very preferably selected such that the ratio of the gap width of the doctor knife to the layer thickness of the mixture applied in step (b), possibly after drying the mixture in step (c), is at least 1:1, more preferably at least 2:1, even more preferably at least 3:1, very preferably at least 4:1 and most preferably at least 5:1. Using a gap width of 30 μm, for example, this can be achieved at an application rate of about 5 mm/s. Accurate adjustment is possible by means of simple routine tests.

The above-defined ratio of gap width to film thickness of the optionally dried layer applied in step (b) (adhesive layer (K)) is also called the “breakdown factor” for the purposes of the invention. It has been found, surprisingly, that particularly advantageous transdermal therapeutic systems can be obtained when the breakdown factor has a value of 2 or more, preferably 3 or more, even more preferably 4 or more and most preferably 5 or more. In such a case, excellent adhesion properties are achieved at only very small layer thickness of the adhesive layer (K), even when the concentration of analgesic present therein is high, e.g. only slightly below its saturation point.

Preferably, the mixture produced in step (a) has a viscosity (determined with a Brookfield viscometer at 25° C., #27, 20 RPM, SSA EtOAc/heptane 87/13) in the range of 1000 to 10,000 mPa·s, more preferably 2000 to 7500 mPa·s and even more preferably from 2500 to 4500 mPa·s.

The mixture prepared in step (a) preferably contains a solvent. In particular, this is an organic solvent that has a boiling point at atmospheric pressure of less than 100° C., more preferably less than 90° C., even more preferably less than 80° C. and most preferably less than 70° C. Ethanol is particularly preferred.

The optional drying step (c) of the process of the invention may be conducted at an elevated temperature and/or reduced pressure. It is preferably conducted under certain conditions and for a certain period of time such that the residue of the solvent used in the adhesive layer (K) after drying is below 1.0 wt. %, more preferably below 0.5 wt. %, even more preferably below 0.1 wt. %, very preferably below 0.05 wt. % and most preferably below 0.01 wt. %.

The above-mentioned breakdown factor is especially influenced by the following factors: the type and quantity of the solvent used, the application rate, the drying conditions, and the viscosity of the mixture. Using simple routine tests, it is possible to determine the values to which the parameters are to be adjusted in order to achieve a certain breakdown factor.

It has been found, surprisingly, that the process of the invention for the production of transdermal therapeutic systems has advantages over the conventional procedures of the prior art which are particularly manifested during the production of extremely thin adhesive layers (K). For example, in the conventional processes used for the production of adhesive layers (K), it is occasionally necessary to adjust the distance between two parallel rollers, between which the sheet of film, one to several meters wide, is guided, to a distance that is of the order of magnitude of a few μm. This imposes particularly high requirements on the surface quality of the two rollers and the alignment of the bearings. Thus, even very small deviations in layer thickness of the adhesive layer (K) of the order of less than 7 μm lead to considerable relative fluctuations in the layer thickness of the adhesive layer (K) over the width of the film sheeting transversely to its direction of travel.

By contrast, the process of the invention has the advantage that no such rollers are necessary but rather the composition that later forms the adhesive layer (K) can be applied by a film-drawing device to a flat surface and settles to a very uniform layer thickness as a result of the evenly acting force of gravity. The drying process also takes place uniformly over the entire surface so that ultimately a very thin adhesive layer (K) can be obtained having, if any, only very small fluctuations in its layer thickness.

The invention also relates to transdermal therapeutic systems that can be obtained by the process described above.

Another aspect of the invention relates to the use of an analgesic, preferably buprenorphine or one of its pharmaceutically acceptable salts or prodrugs, for the production of an above-described transdermal therapeutic system for the treatment of pain. The system of the invention is in this case preferably designed for application over from 1 to 5, more preferably from 2 to 4 and most preferably for 3 or 4 days. In a preferred embodiment, the pain is selected from the group consisting of acute pain, chronic pain, neuropathic pain, visceral pain, nociceptive pain, postoperative pain, phantom pain, headache, especially migraine headache, cluster headache, Herpes neuralgia, stress headache, paroxysmal hemicrania; facial pain, especially trigeminus neuralgia; toothache, jaw pain, contractions, birth pains, delivery pain, pain due to cystitis, ostalgia, backache, lumbago, arthritic pain, burn pain, cardialgia, anginal pain, muscular pain, especially fibromyalgia, abdominal pain, gastralgia, and cancer pain.

In a preferred embodiment, the pain is cancer pain selected from the group consisting of pain from brain tumors, bone cancer, oral cancer, larynx cancer, esophageal cancer, stomach cancer, liver cancer, bladder cancer, pancreas cancer, ovarian cancer, cervical cancer, breast cancer, lung cancer, skin cancer, prostate cancer, adenocarcinoma, basal cell carcinoma, cancer in the gastrointestinal region, skin cancer, and kidney cancer.

The following examples have the purpose of further clarifying the invention but are not restrictive with respect to its scope:

### EXAMPLE 1

Using an Erichsen “Coatmaster 509/MC-1” film-drawing device, 20 g of DURO-TAK® 387-2510 with a solids content of 41% were drawn out to form a film of 30 μm on an RN 75 Hostaphan film. The rate of application was 5 mm/s. After a drying time of 2 hours, the entire surface of the dried adhesive film was covered with a Hostaphan RNT 36 film (siliconized on one side) as a protective layer.

The layer thickness of the RN 75 and RNT 36 Hostaphan films used was determined with the aid of a micrometer screw and was 79 μm and 38 μm respectively. The total layer thickness of the composite was 121 μm (determined with a micrometer screw), the layer thickness of the adhesive layer being 4 μm.

### EXAMPLE 2 (FOR COMPARISON)

As in Example 1, using an Erichsen “Coatmaster 1” film-drawing device, 20 g DURO-TAK® 387-2510 with a solids content of 41% were drawn out to form a film of 30 μm on an RN 75 Hostaphan film. The rate of application in this case, however, was 70 mm/s.

After a drying time of 2 hours, the entire surface of the dried adhesive film was covered with a Hostaphan RNT 36 film (siliconized on one side) as a protective layer.

The layer thickness of the RN 75 and RNT 36 Hostaphan films used was determined with the aid of a micrometer screw and totalled 114 μm. The total layer thickness of the composite was 160 μm, the layer thickness of the adhesive layer being 46 μm.

### EXAMPLE 3

As in Example 1, a composite was prepared from 16 g of DURO-TAK®387-2510 with a solids content of 41% and 4 g of ethanol. The layer thickness of the RN 75 and RNT 36 Hostaphan films used was determined with the aid of a micrometer screw and totalled 118 μm. The total layer thickness of the composite was 122 μm, the layer thickness of the adhesive layer being 4 μm.

### EXAMPLE 4

As in Example 1, a composite was prepared from 16 g of DURO-TAK(387-2510 with a solids content of 41% and 4 g of Carbopol 980/Ethanol-Gel (containing 0.08 g Carbopol 980). The layer thickness of the RN 75 and RNT 36 Hostaphan films used was determined with the aid of a micrometer screw and totalled 116 μm. The total layer thickness of the composite was 121 μm, and the layer thickness of the adhesive layer was 5 μm.

### EXAMPLE 5

As in Example 1, a composite was prepared from 16 g of DURO-TAK®387-2510 with a solids content of 41% and 4 g of 2% hydroxypropylcellulose/ethanol gel (containing 0.08 g hydroxypropylcellulose). The layer thickness of the RN 75 and RNT 36 Hostaphan films used was determined with the aid of a micrometer screw and totalled 116 μm. The total layer thickness of the composite was 122 μm, and the layer thickness of the adhesive layer was 6 μm.

### EXAMPLE 6

As in Example 1, a composite was prepared from 10 g of DURO-TAK(®387-2510 with a solids content of 41% and 10 g of 2% hydroxypropylcellulose/ethanol gel (containing 0.2 g hydroxypropylcellulose). The layer thickness of the RN 75 and RNT 36 Hostaphan films used was determined with the aid of a micrometer screw and totalled 112 μm. The total layer thickness of the composite was 116 μm, and the layer thickness of the adhesive layer was 4 μm.

### EXAMPLE 7

As in Example 1, a composite was prepared from 10 g of DURO-TAK®387-2510 with a solids content of 41% and 10 g of 2% Carbopol 980/ethanol gel (containing 0.2 g Carbopol 980). The layer thickness of the RN 75 and RNT 36 Hostaphan films used amounted to 112 μm. After drying, the total layer thickness of the composite was 116 μm, and the layer thickness of the adhesive layer was 4 μm.

### EXAMPLE 8

The adhesive strength of the patches produced in Examples 1, 3, 4, 5, 6, and 7 was measured with a TestControl material testing machine, type BTC FR2.5TH.D09 supplied by Zwick/Roell. For this purpose, 75% of a 4 cm wide patch was stuck lengthwise to the top stainless steel plate of a movable slide. The slide was locked in a position below the measuring detector. The remaining, unstuck part of the patch (25%) was mounted in the pneumatic jaws of the measuring device in such a way that the patch pointed vertically upwardly. Now the patch was pulled upward off the slide at a speed of 50 mm/min at room temperature. During the peeling process, the slide was automatically advanced so that the patch was always pulled in a vertical direction. The forces were plotted as a function of path length.

The readings are summarized in the following table:

| | | | | Peeling force in | |
|---|---|---|---|---|---|
| | Thickness of adhesive | DURO-TAK ® | Other components | N/4 cm | |
| Ex | layer (K) (after drying) | 387-2510 | of the mixture | measured | average |
| 1 | 6 μm | 20 g | — | 4.0-6.0 | 5.0 |
| 3 | 4 μm | 16 g | 4 g EtOH | 1.6-3.0 | 2.3 |
| 4 | 5 μm | 16 g | 4 g 2% Carbopol | 14-24 | 19 |
| | | | 980/EtOH gel | | |
| 5 | 6 μm | 16 g | 4 g 2% HPC/EtOH | 1.5-3.0 | 2.3 |
| | | | gel | | |
| 6 | 4 μm | 10 g | 10 g 2% HPC/ | 1.2-2.1 | 1.7 |
| | | | EtOH gel | | |
| 7 | 4 μm | 10 g | 10 g 2% Carbopol | 0.6-1.3 | 1.0 |
| | | | 980/EtOH gel | | |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereto.

What is claimed is:

1. A transdermal therapeutic system for administration of an analgesic, comprising:
a carrier layer (T);
an adhesive layer (K) that immediately adjoins the carrier layer (T), is based on a pressure-sensitive adhesive, and contains the analgesic, wherein the layer thickness of the adhesive layer (K) is less than 7.0 μm.

2. The system according to claim 1, further comprising a strippable protective layer (S).

3. The system according to claim 1, wherein the content of said analgesic in said adhesive layer (K) is greater than 0.60 g/m², based on the area of said adhesive layer (K).

4. The system according to claim 1, wherein after application of said adhesive layer (K) to a suitable part of the skin of a patient, at least 20 wt % of the amount of analgesic originally present in said adhesive layer (K) is released within a period of 72 hours.

5. The system according to claim 1, wherein after application of said adhesive layer (K) to a suitable part of the skin of a patient, at least 50 wt % of the amount of analgesic originally present in said adhesive layer (K) is released within a period of 72 hours.

6. The system according to claim 1, wherein said adhesive layer (K) contains said analgesic in a concentration of from 0.1 to 25 wt %, based on the total weight of said adhesive layer (K).

7. The system according to claim 1, wherein the concentration of said analgesic in said adhesive layer (K) ranges from 90% to 100% of the analgesic's saturation point.

8. The system according to claim 1, wherein the layer thickness of said adhesive layer (K) is greater than 2.0 μm and less than 5.0 μm.

9. The system according to claim 1, wherein said analgesic is an opioid.

10. The system according to claim 9, wherein said opioid is selected from the group consisting of fentanyl, alfentanil, sufentanil, phenoperidine, anileridine, remifentanil, morphine, diacetylmorphine, hydromorphone, nicomorphine, oxycodone, diamorphine, ethylmorphine, ketobemidone, pethidine, dextromoramide, piritramide, dextropropoxyphene, bezitramide, methadone, pentazocine, phenazocine, buprenorphine, butorphanol, nalbufine, tilidine, dezocine, meptazinol, (1R,2R)-3-(3-dimethylamino-1-ethyl-2-methylpropyl)-phenol, (2R,3R)-1-dimethylamino-3(3-methoxyphenyl)-2-methylpentan-3-ol, 6-dimethylaminomethyl-1-(3-methoxyphenyl)-cyyclohexan-1,3-diol, and (1R,2R)-3-(2-dimethylaminomethylcyclohexyl)-phenol, or said opioid is a physiologically acceptable salt or prodrug of one of the foregoing.

11. The system according to claim 10, wherein said opioid is buprenorphine or one of its pharmaceutically acceptable salts or prodrugs.

12. The system according to claim 1, wherein the average force which is necessary to peel a 4 cm wide system orthogonally from a flat stainless steel surface at a speed of 50 mm/min is at least 1.0 N/4 cm.

13. The system according to claim 1, wherein said carrier layer (T) has a layer thickness ranging from 5.0 μm to 125 μm.

14. The system according to claim 1, wherein said carrier layer (T) has a layer thickness ranging from 125 μm to 2000 μm.

15. The system according to claim 1, wherein said carrier layer (T) forms one of two surface layers of said system.

16. The system according to claim 1, wherein said pressure-sensitive adhesive, on which said adhesive layer (K) is based, contains a polymer selected from the group consisting of polyacrylates, polyisobutylene, and silicones.

17. The system according to claim 1, wherein said adhesive layer (K) contains at least one adjuvant for the improvement of the transdermal permeation of the analgesic, the adjuvant being selected from the group consisting of surfactants, amines, inorganic bases, polyvinyl pyrrolidone, propylene glycol, benzyl alcohol, menthol, isosorbid nitrate, dodecylazacycloheptan-2-one, lactic acid, and ethanol.

18. A process for producing a transdermal therapeutic system according to claim 1, comprising the following steps:
mixing constituents for an adhesive layer (K) to form a mixture;
applying the mixture onto one of two surfaces of a carrier layer (T); and
drying the mixture after applying the mixture onto one of two surfaces of a carrier layer (T).

19. The process according to claim 18, wherein said step of mixing constituents for an adhesive layer (K) is performed in the presence of a suitable solvent.

20. The process according to claim 18, further comprising the step of:
applying a protective coating (S) to the mixture after applying the mixture onto one of two surfaces of a carrier layer (T).

21. The process according to claim 18, wherein the step of applying the mixture onto one of two surfaces of a carrier layer (T) involves the assistance of a film drawing apparatus, the gap width of a doctor blade of said film drawing apparatus being in the range of from 10 to 50 μm.

22. The process according to claim 21, wherein application rate of the mixture onto one of two surfaces of a carrier layer (T) ranges from 1.0 to 25 mm/s.

23. The process according to claim 22, wherein the application rate is selected such that the ratio of the gap width of said doctor blade to the layer thickness of the mixture applied onto one of two surfaces of a carrier layer (T) is at least 1:1.

24. The process according to claim 18, wherein the mixture has a viscosity ranging from 1,000 to 10,000 mPa·s.

25. The process according to claim 18, wherein the mixture produced in contains an organic solvent having a boiling point of less than 100° C. under ambient pressure.
